(19) United States
(12) Patent Application Publication
(10) Pub. No.: US 2005/0279354 A1
(43) Pub. Date: Dec. 22, 2005

(54) Structures and Methods for the Joint Delivery of Fluids and Light

(76) Inventors: Harvey Deutsch, Los Angeles, CA (US); Anthony Kewitsch, Santa Monica, CA (US)

Correspondence Address: ANTHONY KEWITSCH, 515 OCEAN AVE., UNIT 505-S, SANTA MONICA, CA, 90402, US

(21) Appl. No.: 11/160,320

(22) Filed: Jun. 18, 2005

Related U.S. Application Data
(60) Provisional application No. 60/581,401, filed on Jun. 21, 2004.
(60) Provisional application No. 60/588,573, filed on Jul. 16, 2004.

(51) Int. Cl. A61M 15/00; A61N 5/06

(52) U.S. Cl. 128/200.24; 607/88

(57) ABSTRACT

Guides for intubation which simultaneously transport fluids and light into a body site are tube-like in structure and consist of a hollow cylindrical optical core surrounded on its inner and outer walls by a cladding of lower index of refraction. Materials comprising the optical core are selected such that the optical absorption and scatter are sufficiently small to transport light efficiently over an extended distance as fluid is transferred through the tube interior. Methods of fabrication, light coupling and light delivery using waveguide tubes are disclosed. Particular applications of waveguide tubes in the medical and industrial sectors are described.

### REFERENCE TO RELATED APPLICATIONS

This application relies for priority on provisional application 60/581,401 filed on Jun. 21, 2004 and entitled “Structures and Methods for the Joint Delivery of Fluids and Light,” and on provisional application 60/588,573 filed on Jul. 16, 2004 and entitled “Integrated Light and Fluid Waveguides.”

### BACKGROUND OF THE INVENTION

Structures which transmit fluids (i.e., liquids and gases) or light, but not both, are widely available in many different forms. For instance, medical devices such as catheters, cannulas and endoscopes are constructed of various types of tubing to facilitate the transport or exchange of fluids during medical procedures. The effectiveness of these procedures may be considerably enhanced by developing a straightforward method of delivering illumination through these devices, while retaining their small form factor. Presently, the transport of fluids is effectively achieved by the tubular structure, but the simultaneous transport of light has been achieved in an ad hoc fashion by adding an optical fiber, for example. Optical fibers are susceptible to breakage and add additional complexity and expense associated with coupling light into an extremely small diameter optical fiber. In an attempt to overcome these limitations, fibers have been “bundled” to produce an effective large core waveguide; however, the resulting fiber bundle is bulky and expensive. An effective solution to the problem of transporting both fluid and light within an integrated structure has been elusive.

Prior art medical devices have addressed the need to transmit and in some cases receive light by adding optical fiber or light guides to the medical device. For example, Laerdal Medical Corporation and VitalSigns Inc. market a flexible light wand which is inserted into the endotracheal tube and U.S. patent Application 2002/0108610 A1 by Christopher describes improvements to this light wand. Approaches such as these add steps to an already complex medical procedure, creating reluctance on the part of health care providers to adopt the new device.

Other approaches attempt to incorporate discrete optical fibers into the structure of a tube. U.S. patent Application 2002/0162557 A1 by Simon et al., entitled “Endotracheal Intubation Device (II)”, describe the use of a fiberoptic or chemiluminescent light source which delivers light to an endotracheal tube via a sleeve including optical fibers. U.S. patent Application 2002/0077527 by Aydelotte describes an endotracheal tube in which a fiber optic bundle is integrated into the wall of the tube. Alternately, liquid core waveguides have been used for chromatography to obtain accurate optical measurements of a fluid acting as a waveguide core surrounded by the tubing which acts as the cladding. WO 99/64099 by Leary et al. describe the use of an unclad plastic tube as a light guide. This design has the disadvantage that fluids or tissue in contact with the tube degrade or destroy waveguiding characteristics by causing optical loss, since the core is not optically isolated by the cladding. In addition, clear tubing fabricated of plastic has a typical loss of about 2 dB/cm, so it is ineffective at transmitting light beyond 10 cm. Clear tubing fabricated of glass has adequate light transmission; however, it does not have a low index cladding and lacks sufficient flexibility. The tube waveguide structure is markedly different from these simple tube designs. The subject of this invention is the design and fabrication of novel waveguide structures which guide both light and fluids in an effective, simple and low cost manner.

### SUMMARY OF THE INVENTION

This invention satisfies the requirement to guide light and fluids simultaneously by providing tubing with an annular core surrounded by a low index cladding comprised of the inner and outer surfaces of the tube. This invention describes the design of tubing which acts as a waveguide itself, eliminating the need for optical fiber(s). This is achieved by designing and fabricating rigid or flexible tubing which consists of a hollow cylindrical core of low optical absorption and scatter, surrounded by inner and outer cylindrical claddings of lower index of refraction. The one or more inner chambers can simultaneously deliver fluids without impacting the optical characteristics of the waveguide. In those applications in which properties of the fluid are to be sensed, cladding regions can be selectively removed to facilitate interaction between the fluid and light guides in a highly controllable fashion. This results in several practical advantages. First, it eliminates the need to embed or attach optical fiber to the tubing. Second, the cross section of the tubing core is relatively large in size (approximately 0.5-3 mm thick wall) and NA (about 0.5) compared to a single mode or multimode fiber core (0.01 to 0.05 mm in diameter) with NA's of between 0.12 to 0.5. As a result, the alignment, source beam divergence and spatial coherence requirements to efficiently couple light into the waveguide are relaxed by the use of the tube waveguides disclosed herein. A halogen, incandescent or fluorescent light bulb, chemiluminescent or LED light source may suffice instead of a more costly laser source.

This waveguide structure further offers flexibility in tailoring the spectral characteristics of the illumination to cover a broad spectral range (10's to 100's of nm, for example) of potential importance for spectroscopy. In some situations it is advantageous that the light source include ultraviolet wavelengths for use in locally preventing infection, for example, while at the same time using near infrared wavelengths to locate the end of the device deep within tissue. Light from single or multiple sources of different wavelengths can be efficiently coupled into the tube waveguide because of the large cross section. This use of structured illumination potentially delivered to different spatial locations along the tube allows additional functionality to be realized. In addition, the local removal of the tubing cladding can be used to optically detect the presence of fluids within the waveguide; for example, the light guidance can be compromised if the liquid index of refraction is higher than that of the tubing core. Finally, the high optical intensities local to an optical fiber endface also have the potential to damage tissue, an effect which is reduced by using a large core tube waveguide.

One application of this invention is the delivery of visible illumination to the tip of an endotracheal tube to assist in visualizing the trachea during the intubation procedure. While fiberoptic light wands have been proposed for this purpose, clinical studies have cast doubt on the effectiveness of these techniques because of the increased procedural complexity. To overcome this, we disclose an endotracheal device incorporating a light source coupled to a waveguiding tube which delivers visible light to the distal end of the tube without the need to add an optical fiber or light wand. In another application, infrared light is delivered to the end of a catheter tube such that the light exiting the tube is transmitted through tissue and detected outside of the body. The imaging of the scattered light enables the catheter to be located in the body as it is inserted.

### BRIEF DESCRIPTION OF THE DRAWINGS

FIG. 1 illustrates a waveguiding endotracheal tube attached to a light source at the distal end of the tube;

FIG. 2 illustrates a cross section of the waveguiding endotracheal tube delineating the core and cladding;

FIG. 3 is a schematic of a waveguide tube coupled to an LED light source;

FIG. 4 is a schematic of a waveguide tube coupled to a chemiluminescent light source;

FIG. 5 illustrates the sagital section of tubing for transmitting both fluids and light;

FIG. 6 represents the technique of side coupling illumination into the wall of the tubing;

FIG. 7 represents the technique of end coupling illumination into the wall of the tubing;

FIG. 8 illustrates an end coupled light source illuminating the entire tube endface;

FIG. 9 illustrates a waveguide tube with cladding regions selectively removed to enable localized optical sensing at a series of locations;

FIG. 10 illustrates a first mirrored tubing endface design and angular distribution of outcoupled light;

FIG. 11 illustrates a second mirrored tubing endface design and angular distribution of outcoupled light;

FIG. 12 illustrates a tubing endface design exhibiting total internal reflection;

FIG. 13 is a block diagram of the optical system used to illuminate, detect and track a catheter delivering infrared light to its distal end;

FIG. 14 illustrates the placement of a catheter with infrared locating feature within the vascular system;

FIG. 15 illustrates the placement of a catheter with infrared locating feature within the digestive system;

FIG. 16 depicts cross sections of waveguiding tubes with multiple internal chambers or multiple light guiding cores;

FIG. 17 depicts tubing junctions which separate or combine separate light and fluid flows from or into a merged flow;

FIG. 18 illustrates the precursor sheets comprising a laminated sheet waveguide coupled to one or more optical fibers;

FIG. 19 illustrates the laminated waveguide;

FIG. 20-A illustrates a tube incorporating a laminated waveguide on inner wall of tube and 20-B illustrates a laminated waveguide on outer wall;

FIG. 21 illustrates a system for self-disinfecting tubing using guided UV light;

FIG. 22 illustrates a cross section of waveguiding tube with multiple independent waveguiding cores;

FIG. 23 illustrates a waveguide tube in which irradiation of a uniform tube has reduced the index along the outer walls to produce a waveguide, and

FIG. 24 illustrates the refractive index profile along a sagittal tube section following irradiation.

### DETAILED DESCRIPTION OF THE INVENTION

This invention discloses structures and methods which guide both light and fluids within an integrated structure. The preferred embodiment is a tube with a single inner chamber for fluids and a single core surrounded by a thin cladding coating.

### EXAMPLE

### Visible Light Guided Endotracheal Tube

An endotracheal tube constructed of a “guide element” has the desirable characteristic that illumination at visible wavelengths launched into the proximal end of the tube can illuminate the distal end when initiating intubation. While the endotracheal tube is inserted into the oral cavity, the co-propagating light source illuminates the anatomy around the tip of the tube, enabling the doctor to more conveniently visualize and accurately position the tube within the larynx without having to rely on a separate light source such as a flashlight, which temporary immobilizes a hand potentially needed for other purposes.

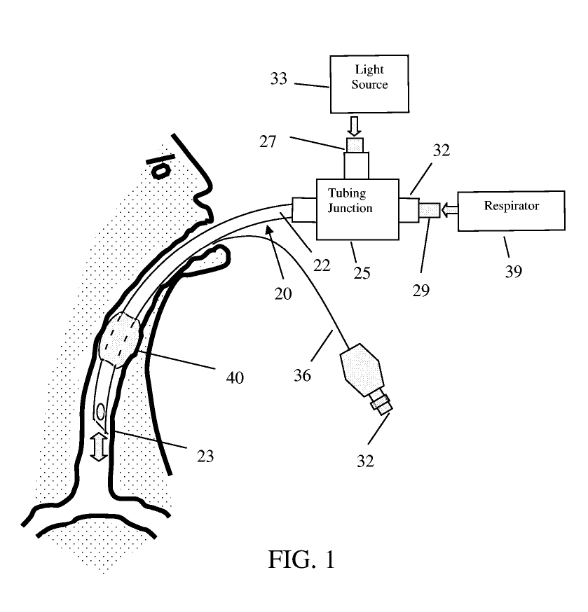

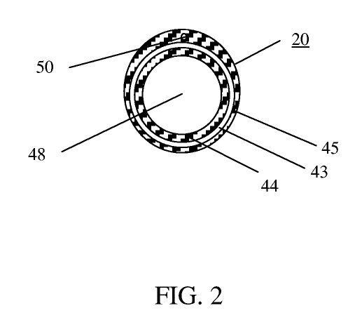

In the preferred embodiment (FIG. 1), a tube waveguide 20 in accordance with the invention is used in an enhanced endotracheal tube device. The tube waveguide 20 consists of a proximal 22 and distal end 23, the proximal end 22 being attached to a tubing junction 25 which includes at least one of a light port 27 and a fluid port 29. The light port 27 transmits a light source 33 from the proximal end 22 to the distal end 23 end of the tube 20. The fluid port 29 may be attached by a quick connect coupling 32 to a respirator 39, for example, to facilitate the exchange of gases. The waveguide tube 20 includes an additional air fluid chamber or passageway 36 connecting cuff 40 via a quick connect coupling 32 which is used to inflate a cuff 40 such that the endotracheal tube forms an airtight seal within the windpipe during respiration. FIG. 2 illustrates a cross section of the waveguide tube 20, delineating the cylindrical core 43 surrounded concentrically by the inner and outer cladding layers 44 and 45 respectively. In this embodiment as an endotracheal tube, a central fluid chamber 48 enables respiration of the patient and an auxiliary chamber 50 in the tube enables the balloon of the cuff 40 to be inflated once the endotracheal device is properly positioned. The outer diameter of outer cladding 45 typically ranges from 5 to 10 mm, and the inner diameter of inner cladding 44 typically ranges from 3 to 8 mm. The core and cladding are fabricated of silicone elastomer with indices of refraction of 1.45-1.44 and 1.44-1.43, for example. The use of silicone provides the requisite tubing transparency (<0.1 dB/cm loss), flexibility, water and heat resistance (to enable sterilization).

### EXAMPLE

### Single Mode Waveguide Tubes For the Radiation

The dimensions of a typical tube waveguide result in multimode waveguiding characteristics for visible and near infrared light. However, waveguides with approximately 1 mm thick guiding regions are expected to be single mode for THz radiation, whose wavelength is on the order of 100 um. THz radiation experiences dramatically less scattering than infrared absorption, so the delivery of THz electromagnetic energy within the body may enable novel medical applications such as deep tissue imaging.

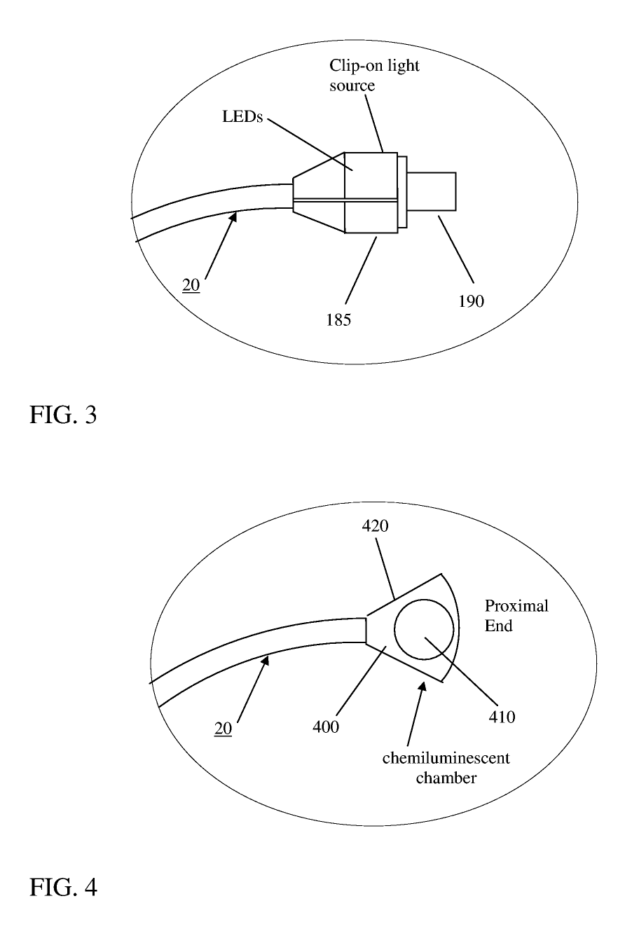

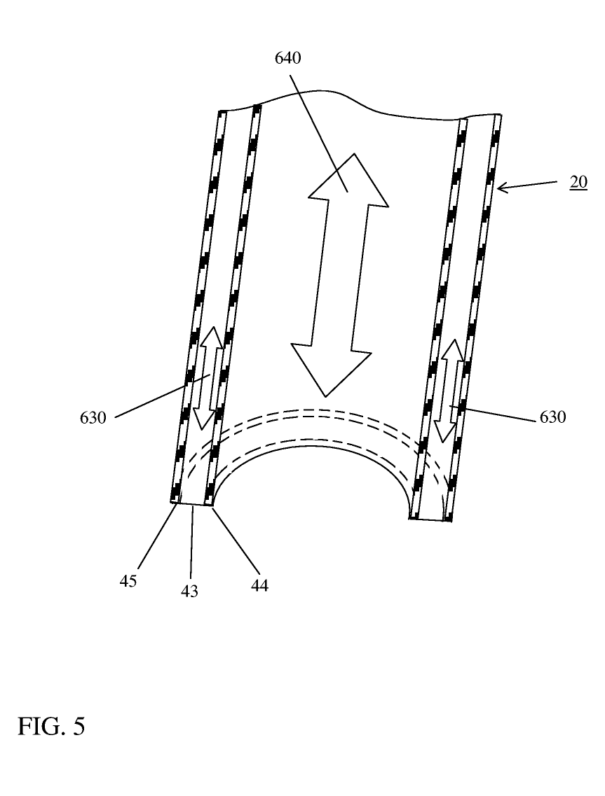

The guide element may be attached to a light source using one of the techniques illustrated in FIGS. 3 and 4. FIG. 5 illustrates a cross sectional view of the tube waveguide 20 transmitting light 630 and fluid 640. The wall of the tubing includes an outer cladding 45 of low index of refraction, a core 43 of high index of refraction, and an inner cladding 44 of low index of refraction. The core is fabricated of silicone elastomer of index of refraction approximately equal to 1.45, and the cladding is fabricated of fluorinated silicone of index of refraction approximately equal to 1.43 by extruding the core and subsequently coating the cladding layers or by co-extruding the core and clad simultaneously. The optical loss for wavelengths from 300 nm to 1400 nm is of the order of 0.1 dB/cm. This siloxane material combination provides superior flexibility, strength and resistance to moisture and high temperature. Typical tubing dimensions are 10 mm outer diameter, 7 mm inner diameter, and 1.5 mm wall thickness. The cladding thickness is in the range of 0.10 to 0.01 mm. The silicone is processed to be free of contamination and voids which may result in scatter and optical loss.

Design and Fabrication

In general, optical waveguides consists of a structure in which a high index, optically transparent core material is surrounded by an optically transparent lower index cladding material. Light within a cone half angle of θ is guided within the high index material through the mechanism of total internal reflection, where the angle θ is given by the expression:

$$NA = \sqrt{{n}_{core}^{2} - {n}_{clad}^{2}} = n sin θ ,$$

and NA is defined as the numerical aperture. The typical geometry is a solid core surrounded by a cladding. Optical fiber waveguides are fabricated from silica glass doped with germanium for the core and potentially boron or fluorine for the cladding. The NA of silica optical fibers is typically in the range of 0.12 to 0.6. Alternately, optical fibers are fabricated of plastic and typically consist of a methacrylate core surrounded by a fluorine doped cladding. The NA of plastic optical fibers is typically 0.5. Both the glass and plastic material systems have been developed to provide ultra-low loss transmission in fibers. Glass optical fiber exhibits loss of about 0.3 dB/km, and plastic optical fiber exhibits a loss of about 10 dB/km. Waveguides are further classified as single mode or multimode. For transmitting high data rate communications, single mode is optimal; however, for efficient delivery of light, multimode waveguides are preferred. Light guides are equivalent to highly multimoded waveguides for visible and near-infrared wavelengths as a result of their large cross sectional areas relative to the wavelength of light.

The design and fabrication of a flexible tube exhibiting superior waveguiding characteristics introduces unique and additional considerations not addressed in the prior art. For instance, there are a limited number of materials suitable for use both as tubing and as an optical waveguide, some of which are described in Table 1. FIG. 5 illustrates a cross sectional view of the tubing waveguide which transmits light 630 and fluid 640. The wall of the tubing includes an outer cladding 45 of low index of refraction, a core 43 of high index of refraction, and an inner cladding 44 of low index of refraction. Suitable materials (e.g., polymer and/or glass) should be selected such that the indices of refraction of the core and cladding are sufficiently different to achieve an NA of greater than 0.1, preferably >=0.5. These material combinations should also be compatible in their physical properties such that stable structures can be produced from these combinations. Furthermore, the materials should exhibit low optical absorption (<0.2 dB/cm) such that a significant fraction of light can be delivered through a 0.5 to 1 meter length. For the same reason, loss due to scattering should also be less than <0.2 dB/cm. This is accomplished by minimizing contaminants and bubbles during the fabrication process. Typical processes to fabricate these structures in plastic are extrusion, molding, and dip coating. Typical processes to fabricate glass waveguide tubes include sol-gel, modified chemical vapor deposition (MCVD), outer vapor deposition (OVD) and IVD (inner vapor deposition).

Tubing is commonly fabricated from silica, polyvinylchloride, polyethylene, polypropylene, Teflon, silicone, or rubber. Materials suitable for waveguiding comprise a subset of materials which exhibit low optical absorption/scatter at the wavelengths of interest (visible or infrared, for example). In many cases, this necessitates that the index of any fillers be matched to the surrounding material so that the tube is not translucent. For applications which require flexible tubing, plastics are preferred to glass (see Table 1). Silicone and the class of siloxanes provide adequately low inherent optical absorption from 300 to 1600 nm. Furthermore, a silicone tubing core index of 1.45 and a fluorinated silicone tubing cladding index 1.35 gives an NA of about 0.5. For applications which require rigid tubing, silica glass is the optimal material. Various doping combinations can be used to achieve an NA of 0.5, for example, the core can be fabricated of germanium doped silica or pure silica, and the cladding can be fabricated of fluorine or boron doped silica or pure silica. In particular, polymer coated glass tubing as in HPLC (high pressure liquid chromatography) and capillary electrophoresis serve as effective waveguiding structures.

TABLE 1
Transparent plastics

| | Index of | |
|---|---|---|
| Material | Refraction | Optical Absorption |
| polymethyl- | 1.496 @486 nm | 0.00014 cm⁻¹ @ 500 nm |
| methacrylate | | |
| | 1.488 @ 656 nm | 0.00035 cm⁻¹@ 670 nm |
| | | transparent to 1600 nm |
| polystyrene | 1.6169 @ 435 nm | 0.00067 cm⁻¹ at 610 nm |
| | 1.5738 @ 1000 nm | 0.00070 cm⁻¹ at 670 nm |
| | | transparent to 1600 nm |
| dimethyl/ | 1.60 to 1.40 | 0.66 dB cm⁻¹ at 1550 nm |
| methylphenyl | | |
| siloxane | | |
| copolymer | | |
| Dow Corning | | 0.62 dB cm⁻¹ at 1550 nm |
| methylphenyl | | |
| siloxane | | |
| Dow Corning | | 0.54 dB cm⁻¹ at 1550 nm |
| fluoro- | | |
| silicone - 1 | | |
| Dow Corning | | 0.35 dB cm⁻¹ at 1550 nm |
| fluoro- | | |
| silicone - 2 | | |
| Dow Corning | | 0.49 dB cm⁻¹ at 1550 nm |
| phenyl resin | | |
| siloxane - 1 | | |
| Dow Corning | | 0.39 dB cm⁻¹ at 1550 nm |
| phenyl resin | | |
| siloxane - 2 | | |
| Dow Corning | 1.46 at 1310 nm | 0.1 dB cm⁻¹ at 500-600 nm |
| OE-4100 | | |
| optical | | |
| elastomer | | 0.1 dB cm⁻¹ at 1310 nm |
| (silicone | | |
| family) | | |

Tubing such as seen in FIG. 5 is inexpensively fabricated by extrusion or molding. The core 43 consists of a siloxane compound such as Dow Corning OE-4100, a material optimized to have low optical absorption in the ultraviolet, visible or near infrared. The cladding 44, 45 consists of a fluorosilicone compound. The core material, if processed to maintain high purity and a low level of trapped bubbles, exhibits good transparency in the UV, visible (0.1 dB/cm) and near-IR (0.1 dB/cm at 1310 nm). The fabrication is advantageously performed in a clean area or clean room environment, and bubbles are reduced by preparing the precursor materials through vacuum degassing, for example. Note that silicone polymers display a wide range of refractive indices, which provide great flexibility in tailoring the optical characteristics for a particular application (e.g. wavelength of operation or NA). Siloxane compounds exhibit excellent resistance to heat and moisture, and can be cured by addition curing with SiH to SiVinyl or ultraviolet light cure. This allows, for example, the sterilization of devices without degrading the optical or mechanical characteristics.

Coupling of Light Into Tube Waveguides

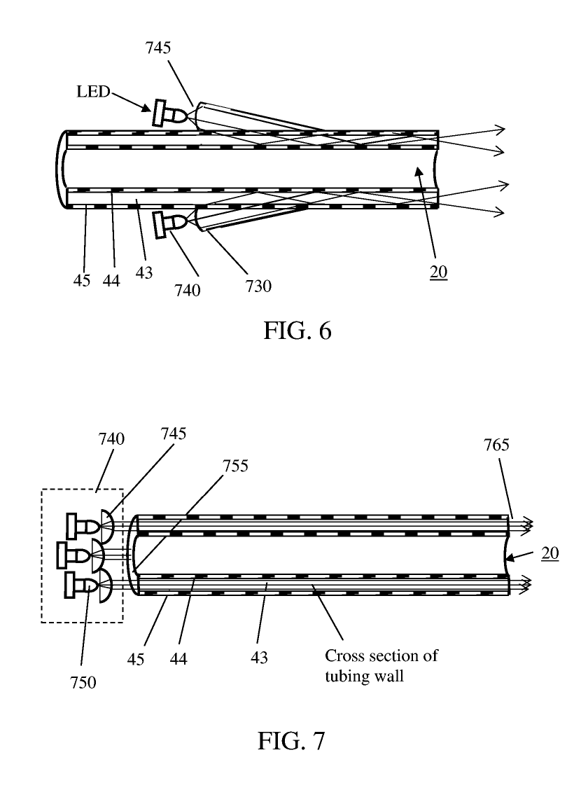

Several approaches to efficiently couple light into the tube are enabled by the high NA and relatively large cross sectional area possible with the tube waveguides. In one example (FIG. 6), a ring-like arrangement of LED's 740 is side coupled into the tubing walls 710 along the full 360 degree circumference. The diverging output of the LED's 740 are collimated by a lens 730 (either integrated into the LED housing or aligned external to the LED) to provide a spot diameter nominally equal to the core 43 thickness of the tubing 20. The resulting half angle of the illumination plus the average angle of the illumination relative to the tubing wall should be less than or equal to the acceptance angle θ of the tubing waveguide core 43, as given by above equation. Note that the larger the NA of the waveguide, the larger the acceptance angle and the higher the coupling efficiency. The thickness of the waveguide determines whether it is multimode or singlemode. In the visible (400 to 700 nm) and near infrared (700 to 1700 nm) wavelength ranges, a waveguide of this type (wall thicknesses of tubing are >=1 mm) is highly multimode. Single mode behavior generally results for waveguide dimensions on the order of λ/NA.

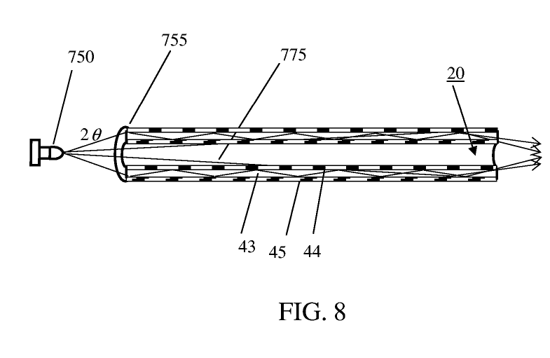

An alternate approach to end coupling the light source is to direct the illumination on-axis into the tubing. In FIG. 7, a ring of LED's 740 is coupled by lenses 745 into the end face of the tube 755. The illumination then forms a ring at the output face 765. Alternately, in FIG. 8, a single domed LED 750 with a beam divergence half angle of θ is coupled to the end face of the tube 755. Those light rays 775 launched on the inside of the tubing eventually get captured by total internal reflection within the core 43 downstream of the proximal end of the tube and become guided in the core 43. Only those rays whose incidence angle is less than the minimum angle for total internal reflection in the waveguide will remain guided in the core 43.

Shaping of Tube Waveguide Endfaces

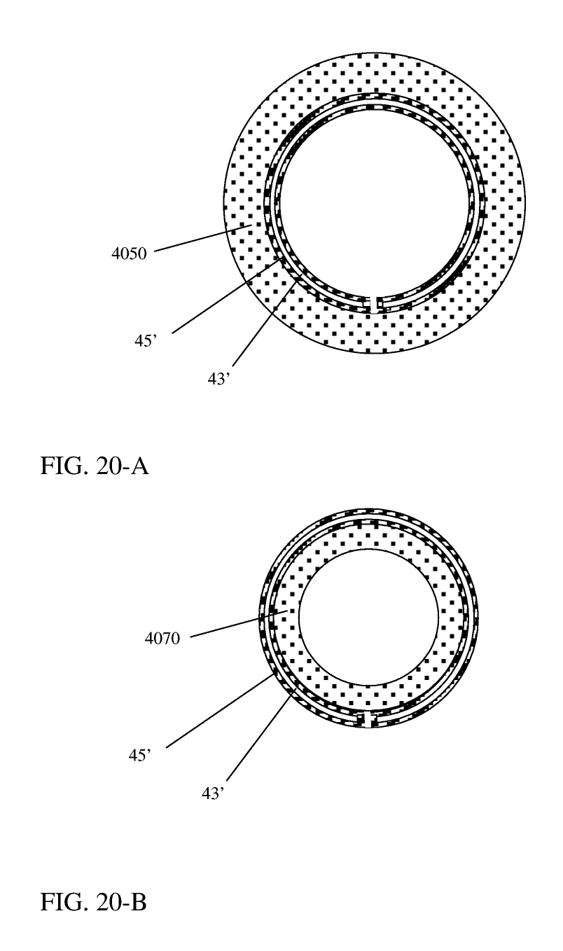

To couple light into the tube or modify the divergence angle at the output, the tubing wall at the point where the tubing is sectioned can be rounded, for example by heating, to form a lens. This may eliminate the need for a coupling lens between the light source and waveguide and also shape the beam focusing/divergence characteristics at the distal end of the waveguide 20 comprised of a core 43 and cladding 44 surrounding the fluid chamber. A “domed” tubing endface serves as a lens. The dome can be concave or convex to provide negative or positive lensing, respectively, to produce a diffuse or localized intensity pattern. Alternately, an azimuthally symmetric dome or dimple may be formed such that the tube endface is half-toroidal in shape. In this configuration, the output of the waveguide produces a “donut” or ring-like output. For optical sensing applications, light can also be emitted from the side of the tube by locally modifying the cladding 44, 45 such that light 1085 is outcoupled from the selected regions of the tube (FIG. 20). This allows spectroscopy to be performed at different locations by use of a network of tubing sensors attached to a light source and detector/spectrometer.

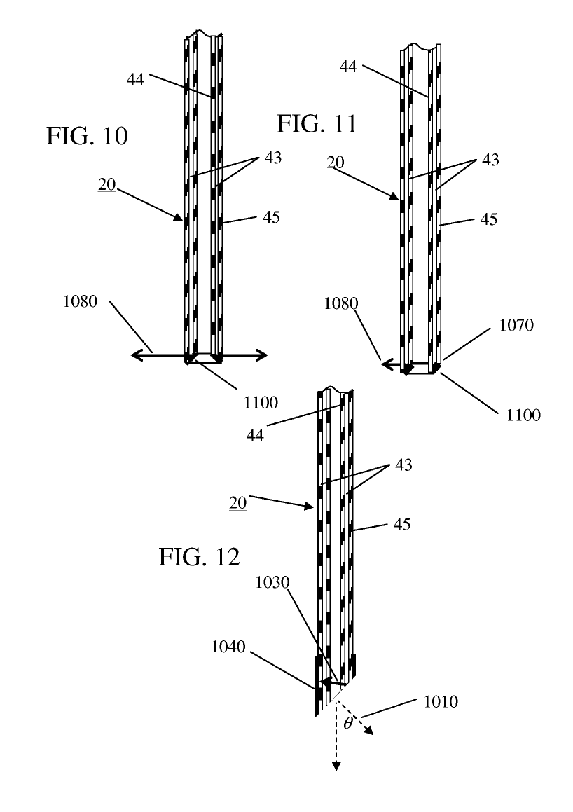

FIG. 10 illustrates an angled endface which has a high reflectivity mirror 1100 on the endface. The mirror directs all the light 1080 normal to the tubing walls 20, such that a full 360 degree fan of illumination 1084 can be produced for the azimuthally symmetric case. For the azimuthally asymmetric case, the light is directed in two beams traveling in opposite directions 1080.

FIG. 11 illustrates a “cleaved” and mirrored endface 1100, which directs light out either in a 360 degree fan for the azimuthally symmetric case, or in a relatively narrow cone normal to the longitudinal axis of the tube in the azimuthally asymmetric case. Note that for the latter configuration, the light 1080 exits the tube only at a particular azimuthal angle. This directionality of emission provides information as to the angular orientation of the element within the body. This orientation information may be important for those tubes which are formed to hold the shape of a natural arc, for example. The arc is advantageous so that the tube conforms to the natural shape within the body while imposing a minimum of restoring force on the walls of the body cavity. However, it may be necessary to determine the orientation of this arc relative to the patient's physiology. Alternately, this orientation information may be necessary to position of the unit such that high power optical pulses can be delivered in a particular direction.

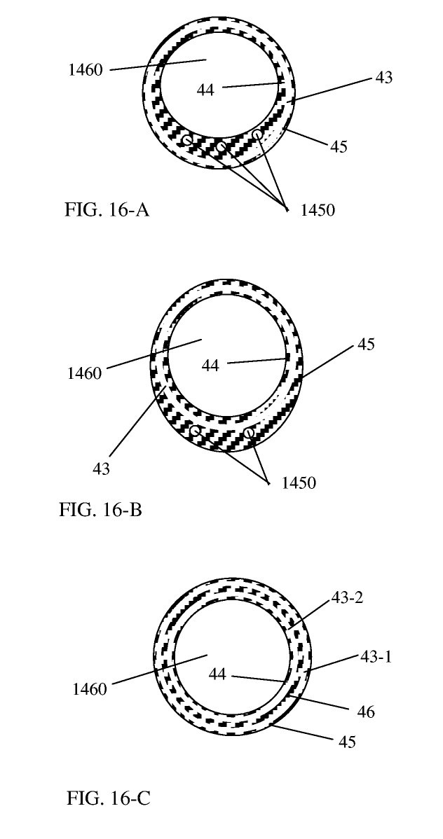

This invention further provides means to transport fluids within one or more chambers of the waveguide. These additional chambers are advantageously formed in the inner or outer cladding so that their contribution to optical loss is minimized, as illustrated in FIG. 16. These structures are comprised of one or more cores 43, 43-1, 43-2 surrounded by claddings 44, 45. A primary chamber 1460 and potentially one or more secondary chambers 1450 transport fluids. As an example, the ability of hollow waveguides to supply or aspirate fluids from within the body is advantageous for a prostatectomy system which requires a flow of fluid while the inserted element delivers sufficient optical energy to ablate the offending tissue. An endotracheal tube includes an air guide for respiration in additional to a guide used to inflate the cuff. A balloon catheter for angioplasty similarly requires an additional chamber to inflate a balloon. Alternatively, a light transmitting element inserted into the eye is used to break down cataracts and simultaneously aspirate the fluid in the eye to remove particle matter formed by ultrasonic agitation. The integration of this fluid and light guiding functionality is not met by present devices.

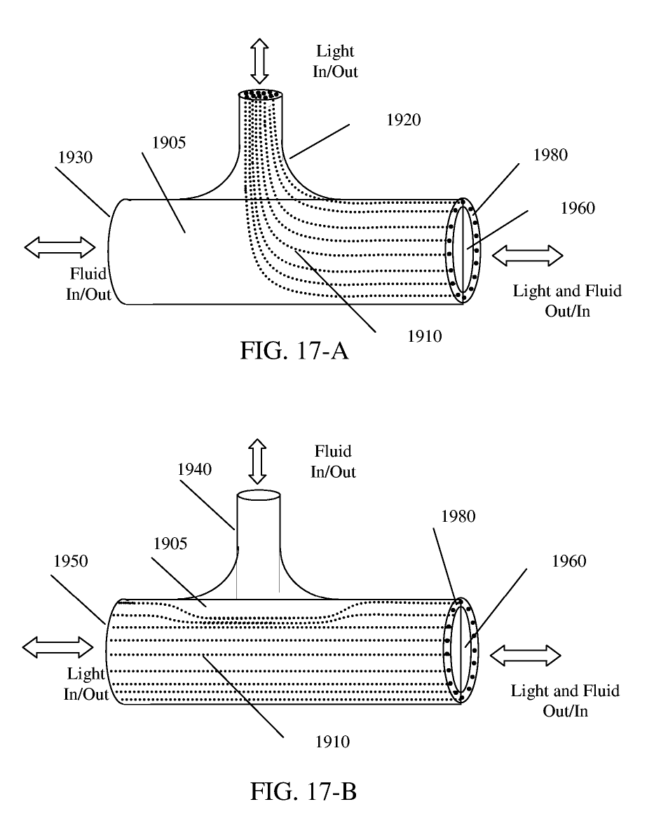

The invention further discloses a tubing junction (FIG. 17) which splits or combines independent fluid 1930, 1940 and light 1920, 1950 conduits into a common fluid/light conduit 1960. Dashed light lines 1910 designate the path of each optical ray, which may be formed by embedding high index guides within the low index matrix 1905. The junction shell is fabricated of a rigid plastic material, and the embedded waveguides are higher index plastic or glass. Junction endface 1980 is coupled to a waveguide tube whose core is aligned with the light lines 1910 at endface 1980. In the preferred embodiment, the three terminations of the tubing junction utilize quick-connect type fittings.

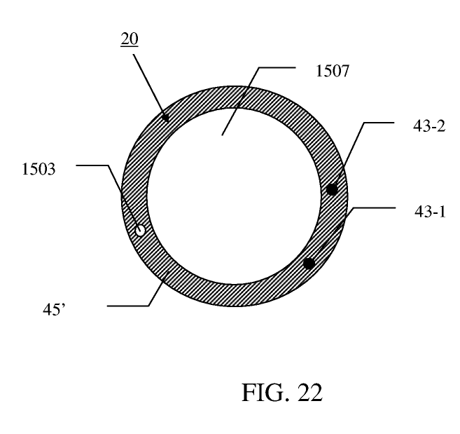

FIG. 22 illustrates a waveguide tube 20 which includes multiple light guiding cores 43-1, 43-2 of nominally round cross section. Chambers 1503 and 1507 are additionally formed in the structure to enable the transfer of fluids. This structure may be formed by extruding silicone or pvc tubing, for example, wherein chambers 1503, 1507, 43-1 and 43-2 are formed in the cladding material. Subsequent to this, material of higher index of refraction relative to the cladding material may be injected into chambers 43-1 and 43-2 to form a high index light guiding region.

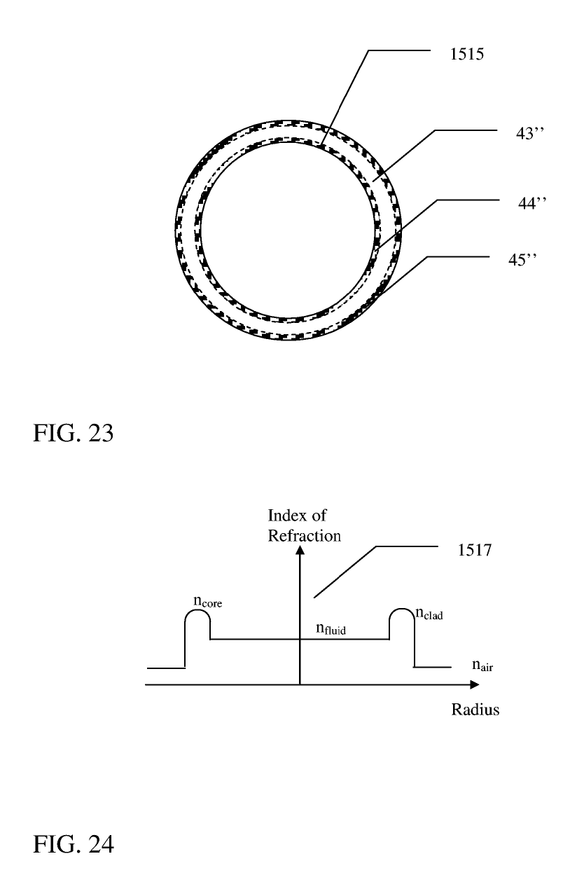

In an alternate embodiment, a waveguide tube may be formed by irradiating tubing of uniform index of refraction n_{core} with gamma ray, electron beam or ultraviolet irradiation such that the exposed inner and/or outer walls of the tube undergo a physical transformation which reduces the index of refraction to n_{clad}. The resulting index of refraction profile 1517 is represented by FIG. 24. The high energy illumination has a limited propagation depth within the tube and a graded refractive index profile is formed. For instance, gamma irradiation is generated by a Cobalt 60 isotope. This irradiation typically interacts with polymers via two mechanisms. The first, chain scission, results in reduced tensile strength, elongation, and reduced index of refraction. The second, crosslinking, results in increased tensile strength, shrinkage, and increased index of refraction. Both reactions occur simultaneously, but depending on the material and additives, one is usually predominant. Clearly, the former mechanism should be dominant to form the reduced index cladding. An example of the waveguide structure which results is illustrated in FIG. 23. The core 43″ is surrounded by an inner 44″ and outer 45″ cladding, wherein the interfaces between the core and claddings are gradual in nature. The index of refraction and depth of the cladding regions may be varied by tailoring the irradiation conditions.

Total Internal Reflection Interlock

This invention further discloses a passive safety interlock design (FIG. 12) utilizing the phenomenon of total internal reflection (TIR), which is necessary for applications involving optical powers in excess of 100 μW to prevent damage to the human eye. The interlock ensures that the guide does not emit light unless it is safely within a body cavity. It is advantageous to realize this functionality using a waveguide tube of core 43 and cladding 44,45 whose distal tip is prepared at an angle such that light 1030 experiences total internal reflection when the tip is in the air, while transmitting very efficiently when the tip is placed in a medium of higher index of refraction relative to air, such as water or blood. The waveguide has an index of typically 1.45, the index of water is 1.33 and the index of air is 1.00. This corresponds to a total internal reflection angle 1010 between the light propagation direction and the surface normal of the waveguide exit face of:

$${θ}_{tir} = {sin}^{- 1} ( \frac{{n}_{outside}}{{n}_{core}} )$$

For this example, the angle is 43.6 degrees in air, and 66.5 degrees in water. Therefore, the waveguide exit face should be angled between 43.6 degrees and 66.6 degrees so that total internal reflection occurs in air but not in water. The outer cladding 45 of the tube 20 near the exit face should be covered with an absorber 1040 so that the backreflected signal 1030 propagating at a large angle to the core-clad interface does not escape from the waveguide. The infrared absorber may be a suitably opaque dye impregnated epoxy coating, for example. Note that the absorber coating 1040 can be replaced with a reflective coating such that the TIR light is reflected back out the input end of the tube. This reflected optical signal can be detected and used as an indicator that the waveguide is properly inserted into fluid. This provides feedback when a tubular catheter or syringe is properly inserted in the blood carrying artery or vein.

Light Sources

Typical narrow emission LEDs with transparent lenses emit with a cone half-angle of approximately 15 degrees (at the −3 dB points of the far field emission pattern). The maximum emitted power of an LED is typically 150 mW. Light bulbs with reflectors can provide similar illumination patterns with up to several hundred Watts of power. Semiconductor laser diodes with hundreds of mW typically emit with a Gaussian spatial mode of 1 μm beam diameter and a divergence half angle of 30 degrees. Other potential light sources include chemiluminescent vials, fiber amplifiers, semiconductor amplifiers and gas, solid state, or excimer lasers. Any of these sources can be driven continuously, or they may be driven such that the intensity is intermittent or periodic for high power optical pulses of short duration. The selection of the appropriate power/duration ratio can eliminate potential tissue damage effects.

The ease in which light can be coupled into the tube waveguide enables the light source to be portable and/or disposable using inexpensive components, such as a battery operated LED (FIG. 3), light bulb, or chemiluminescent light source (FIG. 4). This eliminates the need to re-sterilize the light source if it is re-used. A low cost LED source 185 can be attached to one end of the waveguide tube 20 to direct light to emit from the distal end. A disposable source such as a chemiluminescent vial 410 also can be coupled to the tube by using a reflective coupling structure 420 which efficiently directs the highly diverging light from the chemiluminescent source 400 into the tubing walls, as illustrated in FIG. 4. The preferred chemiluminescent light source is the high intensity, short duration type (1-5 minutes of emission). The chemiluminescent approach is particularly well suited for medical devices used in the field, where a rugged light source with long shelf life and low weight provides great advantages.

### EXAMPLE

### Smart Tubing

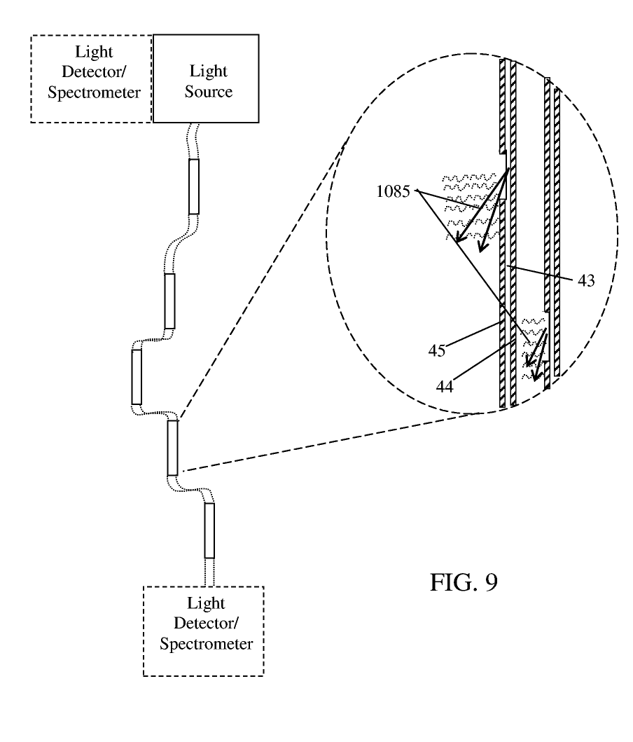

Waveguide tubing serves as “smart tubing” by incorporating sensors which interface the fluid and light conduits. For example, the cladding can be locally removed (FIG. 9) to form a “window” such that light locally samples the fluid at one or more locations along the tubing. The evanescent overlap of the light within the core and fluid in contact with the core allows, for example, the absorption spectrum of the fluid to be monitored by directing the waveguided light into an optical spectrometer. Alternately, sensors can be placed at various locations along the inner and outer cladding of the tube, and these sensors can be interrogated by an optical signal. The optical transmission characteristics of these “windows” can be spectrally controlled by utilizing different coatings such that illumination of different wavelengths can be emitted or detected from different locations along the tube. The use of structured illumination; that is, light whose spectral characteristics are manipulated on a wavelength by wavelength basis, allows particular spectral components to be emitted from different locations along the tube. For example, in medical applications where tubing penetrates the skin, the delivery of ultraviolet light in a ring-like spatial distribution at the point of entry is advantageous to prevent infection. At the same time, it may be desirable to deliver near infrared light for deep tissue imaging at the end of the tube. The natural wavelength dependence of scattering can ensure that short wavelength ultraviolet is scattered into the tissue at the beginning of the tube while longer wavelength infrared light is able to propagate further down the tube.

### EXAMPLE

### Laminated Tube Waveguide Structure

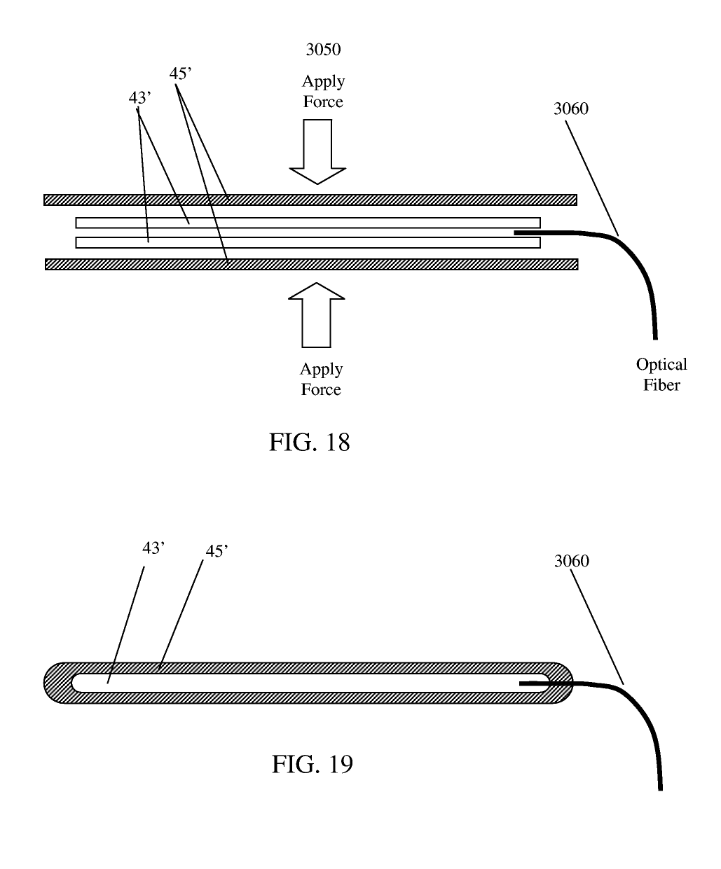

For many applications the tubing material selection is constrained by factors such as weight, strength, environment, and type of fluid being transported. The primary tubing constituent may therefore not be optically transparent. In these situations a preferred approach to designing waveguide tubing is to first produce a sheet-like laminated structure comprised of a core and cladding as illustrated in FIG. 18. The sheet can then be applied to the inner and/or outer diameter of the tube to form an integrated tubing waveguide. The upper and lower sheets ultimately form the low index cladding 45′, and the one or more middle sheets ultimately form the high index core 43′. The use of two core sheets allows inexpensive and efficient coupling from one or more optical fibers 3060 whose NA is equal to or lower than that of laminated structure. By applying heat and pressure 3050, these layers are joined together such that the fiber 3060 becomes locally embedded within the ultimate tubing core 43′, as illustrated in FIG. 19. As illustrated in FIG. 20, this sheet is then applied to either the inner (20-B) or outer (20-A) diameter of the tube 4050, 4070 such that the resulting structure propagates both light and fluid. The FIG. 20-A structure consists of a tube 4050 in contact with the cladding 45′, which surrounds the core 43′ and carries fluid within the central cavity. The FIG. 20-B structure consists of a tube 4070 which carries fluid within the central cavity, attached to the cladding 45′ which surrounds the core 43′. Note that the “seam” or location where opposite ends of the original sheet meet at a particular azimuthal location along the tube 4050, 4070 interrupts the azimuthal symmetry of the waveguide. Since the core thickness is typically much smaller than the uncoiled width of the waveguide, the optical propagation characteristics are essentially unchanged.

This fabrication approach is advantageous for a wide range of tubing and pipe applications of microscopic to macroscopic dimensions. These applications include large pipes such as water, gasoline or natural gas mains, tubes for carrying toxic gases in semiconductor fabrication facilities (e.g., arsine or silane), flammable gases/liquids such as hydrogen or high pressure oxygen in refineries or chemical processing facilities, high pressure hydraulic and fuel lines in aircraft, radiator hoses in automobiles and cooling water lines in nuclear power plants. These examples are for illustrative purposes only. It should be appreciated that a great number of applications benefit from the ability to communicate light along the tube in part because the mechanical integrity of the tube (and as a result the waveguide) can be readily monitored through transmitted or reflected light analysis. The presence of cracks can be detected before the fluid transport properties are compromised. For instance, a local crack in a pipe would produce a crack in the waveguide core which leads to backscattered light. The strength and origin of this scattered light may be monitored quite simply with an optical time domain reflectometer (OTDR) or optical coherence domain reflectometer (OCDR). These instruments are commercially available from Agilent Inc. or Exfo Inc., for example, with a dynamic range in excess of 90 dB and spatial resolution as low as 50 μm.

Alternate approaches to embed sensors in structures utilize optical fiber; however, the effectiveness of these techniques are practically limited by the relatively low number of sensors which can be embedded in the tube. The use of a waveguide tube offers a continuous network of sensors to be distributed along the structure.

### EXAMPLE

### Self-Disinfecting Tubing

For many fluid transport applications it is desirable that the inner chamber(s) of the tube remain free of bacteria. Waveguide tubing allows actinic or ultraviolet radiation to be propagated down the tubing such that the radiation inhibits or destroys bacteria within the tube. The coupling of uv light out of the core and into the fluid is achieved, for example, by introducing a selected level of scatterers within the waveguide core or by locally removing the cladding. The selection of appropriate optical characteristics of the inner and outer claddings enables light to be scattered from the outer wall, the inner wall, or both.

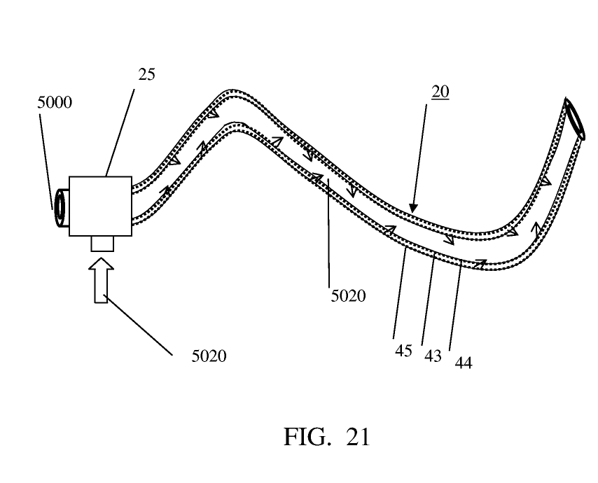

FIG. 21 illustrates the propagation of UV light 5020 down a waveguiding tube 20, in which the waveguide core/cladding characteristics are designed such that UV light 5020 is scattered into the fluid carrying chamber along the length of the tube. UV light 5020 is launched into a tubing junction 25 and the fluid chamber is interfaced to the fluid port 5000. Note that the cladding is not explicitly shown because it is typically of microscopic thickness. UV light of sufficiently short wavelength (<400 nm) is known to inhibit the growth and destroy most forms of bacteria. This type of tubing may find application in ensuring the supply and distribution of bacteria free water in homes or hospitals. Alternately, any of the numerous medical procedures in which tubing is inserted into the human body would benefit from the self-sterilizing nature of UV excited waveguiding tubes.

### EXAMPLE

### IR Light Guided Catheter

A nurse or doctor have no direct feedback regarding the location of the catheter tip when inserting a tube-like catheter into a vein or artery in the absence of a relatively expensive fluoroscopy procedure. This leads to a higher incidence of errors in the placement of the catheter and possible serious medical complications. Bard Inc. had introduced a CathTrack™ catheter locating system based on electronic detection which was not commercially successful because the limited spatial resolution and inconvenience of usage. Alternately, fluoroscopy or ultrasound imaging techniques may provide a real time image of the catheter location; however, these systems are cost prohibitive in most situations. Today, a post implantation x-ray is performed after catheter insertion to confirm catheter tip location and to ensure that the catheter is not being pinched by the clavicle or ribs. This provides a location accuracy of about +/−1 cm. Approaches using near infrared imaging have the potential of eliminating the need for an x-ray.

Wilson and Schears disclosed in Patent Application WO 02/103409 A2 a catheter including an optical fiber illuminated at 780 nm such that catheter is visualized with night vision goggles through tissue. However, this approach is inadequate for several reasons. At 780 nm, tissue causes significant light scattering, which limits the penetration depth of the light. This effect is usually dominated by Rayleigh scattering, wherein the scattering coefficient decreases as the inverse wavelength cubed. Operation at longer wavelengths reduces scattering and leads to improved signal to noise (SNR) ratio at the imager. Furthermore, the use of night vision goggles at near infrared wavelengths as disclosed in WO 02/103409 A2 provides relatively poor sensitivity compared to InGaAs focal plane arrays.

The concept disclosed herein includes a catheter locator system meeting the requisite performance by incorporating waveguide tubing illuminated by wavelengths greater than 1000 nm. In the preferred embodiment, a 1310 nm semiconductor laser diode with 10 to 100 mW optical power is launched into a waveguiding catheter tube with high coupling efficiency. The optimal power is selected such that adequate signal strength is received by the imaging array, outside of the body, while maintaining a local intensity level below the tissue damage threshold. This control is achieved by way of an electronic feedback loop which controls the laser power output such that the received signal achieves a target value. The scattering angle within a blood carrying vein or artery is large enough that a significant amount of light is detected perpendicular to the nominal exit angle of light from the waveguide. Therefore, even though the catheter tip lies approximately parallel to the sagittal plane of the patient, a detectable amount of light is scattered normal to the sagittal plane. An imaging array aligned normal to the sagittal plane can then detect the near infrared light. Further increase in the detected signal can be achieved by angling and reflectively coating the tube endface such that the illumination 1080 is directed more efficiently out of the sagittal plane, as illustrated in FIGS. 10 and 11.

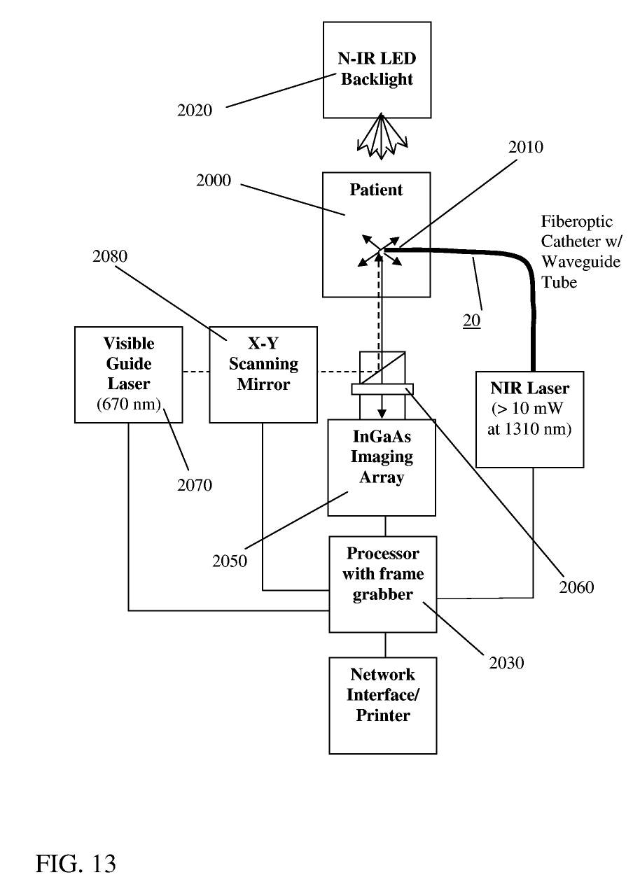

A further element of the invention is a system to track and visualize the catheter, as illustrated in FIG. 13. The signal-to-noise ratio (SNR) is enhanced by blocking wavelengths other than the guiding laser (and possibly the visible tracking laser) with a narrow bandpass filter 2060 placed in front of imaging array. This filter is preferably a multiple cavity thin film interference filter with a passband width of 1 to 10 nm. Note that the higher the imaging system SNR, the thicker the tissue through which the marker can be located. To enable an image of the patient 2000 to be acquired such that the catheter marker 2010 is superimposed, a 1310 nm LED 2020 may be used to backlight the patient 2000 during catheter tracking, such that a frame grabber 2030 connected to a printer captures a reflected infrared light image of the patient's upper chest, including a marker 2010 indicating the position of the catheter 2040. For example, the detector is an uncooled InGaAs detector array 2050 with 320 by 240 pixels. A cooled detector lowers the detector noise floor so that even weaker signals may be detected; however, this performance may not be necessary nor justify the added cost. In addition, it may be desired to include a visible CMOS or CCD imager in the visible, such that the visible and infrared images can be merged.

Since infrared light is not visible to the human eye, a visible indicator of the infrared marker in relation to the patient should be provided to the user. An additional element of the invention is the technique to visualize the infrared image in a manner which augments the normal visual field of view. By applying signal processing techniques (for example, automatically locating the near-ir spot and determining the centroid of the scattered light), a visible laser marker 2070 deflected by a two-dimensional scanner 2080 can be directed onto the body at a location on the skin closest to the internal catheter tip 2090. Alternately, the merged IR and visible wavelength information can be presented on a monitor or projected on a partially transmissive mirror in the light of sight of the doctor.

This approach combines the near infrared image with the normal visual information in a hands-free fashion, functionally providing a “heads-up” type display. As a result, this system does not distract the doctor or nurse and does not require additional training or a change in procedure. Furthermore, as the catheter is directed into certain areas of the body, the light path out of the body may be partially occluded by ribs. This can be extracted by signal processing in a manner such that the marker laser does not disappear each time the catheter passes behind a rib. In addition, the scanning system can not only mark the catheter or endpoint, but also trace out all earlier locations of the probe tip so that the entire catheter is “visualized.” Alternately, light can be emitted simultaneously from several locations along the tube by suitable removal or processing of the cladding.

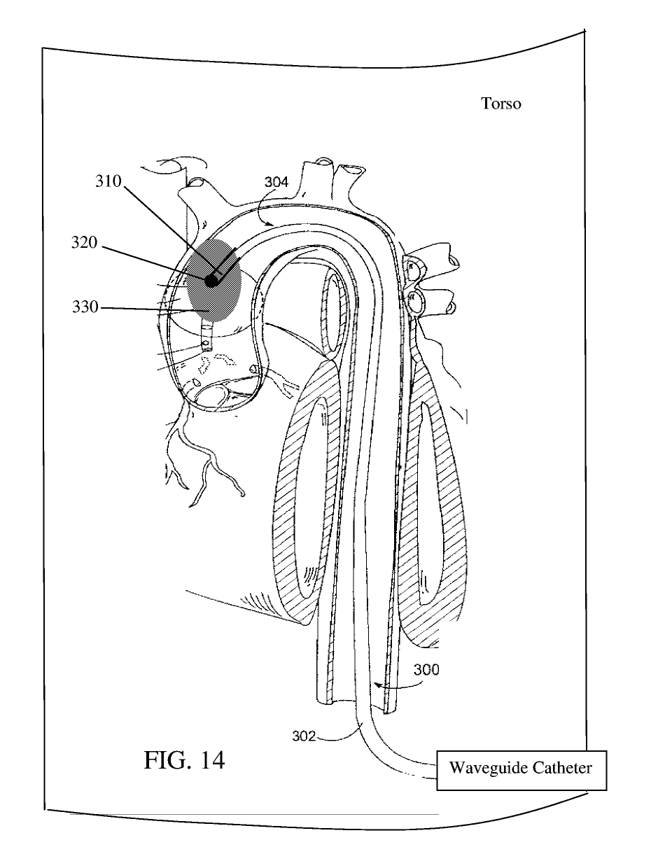

The use of an infrared light marker to locate the catheter within a patient has applications to infusion, cardiovascular, renal, hemodynamic, monitoring and neurological catheters. For example, FIG. 14 illustrates the use of an infrared guiding catheter 304 inserted in the vicinity of the heart. The waveguiding catheter 302 is inserted into the aorta 300. Near infrared illumination exits from the tip of the catheter 310 and is scattered as it exits the body to form an extended spot 330. Image processing is used to infer the exact location of the catheter tip based on the extended scattered light. A visible alignment laser is then directed onto the body via the optical scanner assembly to indicate the catheter tip location 320.

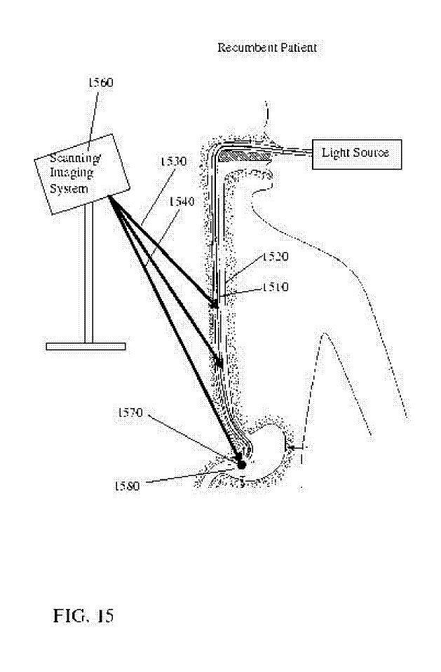

FIG. 15 illustrates the use of an infrared guiding nasogastric tube 1510 within the esophagus 1520. The scanning system 1560 sequentially illuminates the location of the tube by tracing out paths 1530 through 1540 along the torso with a visible alignment laser. The final position of the tip 1570 is determined by processing the diffuse scattered infrared light 1580. It should be apparent that this infrared marker technique is also of value in a single intravenous (IV) procedure or when drawing blood through a syringe (which can be fabricated from a small diameter waveguide tube).

Those skilled in the art will readily observe that numerous modifications and alterations of these devices may be made while retaining the teachings of the invention. Accordingly, the above disclosure should be construed as limited only by the metes and bounds of the appended claims.

1. An intubation device for propagating light energy and fluid internally into the body, the device comprising:
an elongated tubular element pliable enough to conform to a nonlinear pathway within the human body, the tubular element having an optical transparent annular core wall of selected refractive index, and also including cladding material of a different, lower refractive index on both inner and outer sides thereof;
at least one light source optically coupled to transfer optical energy to the tubular element and along the annular core, and
a fluid source coupled to flow fluid along the interior of the tubular element into the body.

2. A device as set forth in claim 1 above, wherein the optical core of the tubular element has a radial thickness of 0.5 to 3.0 mm and a numerical aperture of 0.12 to 0.5.

3. A device as set forth in claim 1 above, wherein the optical material of the tubular element is selected from the class of materials comprising glass and plastics.

4. A device as set forth in claim 1 above, wherein the at least one light source comprises a source of electromagnetic wave energy in the wavelength range from infrared to ultraviolet, and wherein the light source is positioned to launch light energy along the axis of the annular core axis of the tubular element from an end thereof.

5. A device as set forth in claim 1 above, wherein the tubular element has a distal inserted end, and wherein the distal end includes an optical device configured to propagate light energy in a selected pattern from the distal end.

6. A device as set forth in claim 5 above, wherein the optical device at the distal end is configured to propagate light omnidirectionally.

7. A device as set forth in claim 5 above, wherein the optical device at the distal end is configured to propagate light energy toward a focal point.

8. A device as set forth in claim 5 above, wherein the optical device at the distal end is configured to propagate light energy in a pattern along a selected azimuth relative to the direction of light energy propagated along the tubular element.

9. A device as set forth in claim 1 above, wherein the device is adapted for use in endotracheal procedures and also comprises also an inflatable cuff disposed about the exterior of the tubular element in an intermediate position when inserted into the trachea, a fluid conduit along the tubular element coupled at a distal end to the inflatable cuff, and a pneumatic fluid pressure source coupled to the other end of the conduit for expanding the cuff against the trachea.

10. A device as set forth in claim 9 above, wherein the tubular element incorporates the conduit as an interior fluid channel, and wherein the device further comprises a detachable coupling between the channel and the fluid pressure source, and wherein the device also includes a second quick connect coupled to the interior of the tubular element and a respiration source coupled to the second quick connect.

11. A device as set forth in claim 10 above, wherein the outer diameter cladding ranges from 5-10 mm and the inner diameter cladding ranges from 3-8 mm, wherein the core and cladding comprise a silicone elastomer with index of refraction of 1.44-1.45 and 1.44-1.43 respectively, and the silicone elastomer has a transparency of less than 0.5 dB/cm loss.

12. A device as set forth in claim 1 above, wherein the tubular element comprises at least one interior passageway disposed longitudinally therealong in the core/cladding structure.

13. A device as set forth in claim 12 above, wherein the at least one longitudinal passageway is disposed in the core of the tubular element.

14. A device as set forth in claim 12 above, wherein the at least one longitudinal passageway is in the cladding of the tubular element.

15. A device as set forth in claim 1 above, wherein the light source is an ultraviolet source, and wherein the inner cladding is configured to scatter ultraviolet energy internally within the tubular element such as to disinfect the tubular element 19) A device as set forth in claim 1 above, wherein the tubular element includes a side-mounted junction.

16. A device set forth in claim 1 above, wherein the tubular element includes at least one sensing window in the a portion of a cladding layer comprising a localized open volumetric area of the cladding through which light energy transmitted along the core and responsive to the absorption spectrum of the adjacent fluid is directed through the sensing window, and further including an optical sensor disposed in the path of light energy transmitted through the window.

17. A device as set forth in claim 16 above, wherein the tubular element includes a number of sensing windows in the cladding, wherein the windows are disposed along the tubular element, and each further includes a wavelength specific light energy signal responsive element.

18. A device as set forth in claim 1 above, wherein the device is configured to block transmission of light energy at potentially high harmful levels unless the distal end is within a human body passageway, and wherein the distal end of the tubular element is at an angle within a range such that light transmitted along the core is internally reflected when the index of refraction of the surrounding environment is substantially less than that of body fluids.

19. A waveguide for propagating lightwave energy along a path defined by a hollow tubular element that includes a transmissive cylindrical core bounded on each of its inner and outer sides by a cladding of a lower index of refraction to form a lightwave structure through which wave energy is propagated, the combination including at least one cladding window for modifying wave energy.

20. A waveguiding device for propagating electromagnetic wave energy along a propagation path within the human body wherein the device including an annular hollow structure of optical material for insertion in the body, the annular hollow structure having index of refraction variations that propagate light energy therein to a distal end, and a distal end which is angled to provide total internal reflection, except in the presence of bodily fluids which enable light energy to exit distal end of waveguiding device.
